(19) United States
(12) Patent Application Publication
(10) Pub. No.: US 2014/0242196 A1
(43) Pub. Date: Aug. 28, 2014

(54) METHOD OF TREATING VENOMOUS BITES

(71) Applicant: UMM AL-QURA UNIVERSITY, Makkah (SA)

(72) Inventor: SAEED SAEED K. ALGHAMDI, MAKKAH (SA)

(73) Assignee: UMM AL-QURA UNIVERSITY, MAKKAH (SA)

(21) Appl. No.: 13/779,680

(22) Filed: Feb. 27, 2013

(51) Int. Cl. A61K 36/00

(52) U.S. Cl. CPC A61K 36/00
USPC 424/725

(57) ABSTRACT

The method of treating venomous bites, such as those from snakes and scorpions, involves treatment by the administration of a therapeutically effective amount of an aqueous extract of the fruit of Juniperus phoenicea, or pharmaceutically acceptable salts thereof. In order to treat the bites of venomous snakes, scorpions and the like, approximately 500 ml of an extract of the fruit of Juniperus phoenicea, or pharmaceutically acceptable salts thereof, is orally administered to a patient three times daily for a period of five days. In order to make the antivenin, formed as an extract of the fruit of Juniperus phoenicea, a quantity of the fruit of Juniperus phoenicea is ground, and then mixed with water. The mixture is then filtered to separate out the extract of Juniperus phoenicea, the filtrate forming the antivenin.

### BACKGROUND OF THE INVENTION

1. Field of the Invention

The present invention relates to antivenins, and particularly to a method of treating venomous bites, such as snake bites and scorpion stings, by the administration of a therapeutically effective amount of an extract of Juniperus phoenicea for the treatment of venomous bites.

2. Description of the Related Art

Antivenins are biological products used in the treatment of venomous bites or stings. Antivenin is typically created by “milking” venom from the desired snake, spider, scorpion or insect. The venom is then diluted and injected into a horse, sheep or goat. The subject animal will undergo an immune response to the venom, producing antibodies against the venom's active molecule, which can then be harvested from the animal's blood and used to treat envenomation. Due to the difficulty in making antivenins by this process, which involves removal of the venom from a living animal and then injection of the venom into another animal to invoke an immune response, it would be desirable to develop an antivenin which could be easily and quickly produced in mass quantity.

Thus, a method of treating venomous bites with an antivenin and a method of making the same solving the aforementioned problems is desired.

### SUMMARY OF THE INVENTION

The method of treating venomous bites, such as snake bites and scorpion stings, involves the administration of a therapeutically effective amount of an extract of Juniperus phoenicea, or pharmaceutically acceptable salts thereof, for the treatment of venomous bites. In order to treat the bites of venomous snakes, scorpions and the like, approximately 500 ml of an extract of the fruit of Juniperus phoenicea, or pharmaceutically acceptable salts thereof, is orally administered to a patient three times daily for a period of five days.

In order to make the antivenin, formed as an extract of the fruit of Juniperus phoenicea, a quantity of the fruit of Juniperus phoenicea is ground, and then mixed with water. The mixture is then filtered to separate out the extract of Juniperus phoenicea, which forms the antivenin.

These and other features of the present invention will become readily apparent upon further review of the following specification.

### DETAILED DESCRIPTION OF THE PREFERRED EMBODIMENTS

In order to treat the bites of venomous snakes, scorpions and the like, approximately 500 ml of an aqueous extract of Juniperus phoenicea, or pharmaceutically acceptable salts thereof, is orally administered to a patient three times daily for a period of five days. Experiments on both animals and human volunteers has shown the efficacy of the extract of Juniperus phoenicea as an antivenin for both venomous snakes and scorpions, including the Naja melanoleuca, Bitis gabonica, Bitis arietans, Narrow-banded krait (Bungarus multicinctus), Cobra snake (Naja atra, or Chinese Cobra), King Cobra (Ophiophagus hannah), Agkistrodon acutus, Vipera russelli siamensis, Bungarus fasciatus, Agkistrodon blomhoffii, Trimeresurus mucrosquamatus, Trimeresurus stejnegeri, Acanthophis laevis, Hadogenes troglodytes, and the Leiurus quinquestriatus. Toxicological study has shown that the extract of Juniperus phoenicea is safe for human consumption.

The Juniperus phoenicea, or Phoenicean Juniper, is a juniper found throughout the Mediterranean region, from Morocco and Portugal east to Italy, Turkey and Egypt, south on the mountains of Lebanon, Israel, Jordan, and in western Saudi Arabia near the Red Sea, and also on Madeira and the Canary Islands. The tree's essential oil is rich in the tricyclic sesquiterpene known as thujopsene. Preferably, the extract containing the tricyclic sesquiterpene thujopsene is made from the fruit of the Juniperus phoenicea.

In order to make the antivenin, a quantity of Juniperus phoenicea is ground, and then mixed with water. As noted above, preferably the fruit of the Juniperus phoenicea is used. After extraction in water, the mixture is filtered to separate out the aqueous extract of Juniperus phoenicea, which forms the antivenin. For an exemplary initial quantity of 20 g of the fruit of the Juniperus phoenicea, approximately two liters is used to form the mixture. The mixture is left to extract at room temperature for approximately four hours prior to the filtration. As an alternative to simply allowing the mixture to sit for four hours, the mixture may be warmed to a temperature of approximately 50° C. for ten minutes to obtain the extract by heat, followed by filtering and collecting the filtrate.

It is to be understood that the present invention is not limited to the embodiments described above, but encompasses any and all embodiments within the scope of the following claims.

1. A method of treating venomous bites, comprising the step of administering to a patient a therapeutically effective amount of an aqueous extract of Juniperus phoenicea or pharmaceutically acceptable salts thereof for the treatment of venomous bites.

2. The method of treating venomous bites as recited in claim 1, wherein the therapeutically effective dosage is administered to the patient orally.

3. The method of treating venomous bites as recited in claim 1, wherein the step of administering comprises administering about 500 ml of the extract to the patient three times per day.

4. The method of treating venomous bites as recited in claim 3, wherein the extract is administered to the patient for a period of five days.

5. A method of making an antivenin for the treatment of venomous bites, comprising the steps of:
grinding a quantity of Juniperus phoenicea;
mixing the ground Juniperus phoenicea with water to form a mixture of water and ground Juniperus phoenicea;
heating the mixture of water and ground Juniperus phoenicea at a temperature of about 50° C. for about ten minutes;
filtering the heated mixture of water and ground Juniperus phoenicea to provide a filtrate; and
collecting the filtrate, the filtrate forming the antivenin for the treatment of venomous bites.

6. (canceled)

7. (canceled)

8. The method of making an antivenin for the treatment of venomous bites as recited in claim 5, wherein said step of grinding a quantity of Juniperus phoenicea consists of grinding fruit of Juniperus phoenicea.

9. An antivenin for snake bites and scorpion stings, comprising an aqueous extract of the fruit of Juniperus phoenicea, the aqueous extract of the fruit of Juniperus phoenicea made by the process of:
grinding a quantity of Juniperus phoenicea;
mixing the ground Juniperus phoenicea with water to form a mixture of water and ground Juniperus phoenicea;
filtering the mixture of water and ground Juniperus phoenicea to provide a filtrate; and
collecting the filtrate, wherein
the mixture of water and ground Juniperus phoenicea is heated at a temperature of about 50° C. for about ten minutes prior to said step of filtering the mixture.
